(19) United States
(12) Patent Application Publication
(10) Pub. No.: US 2025/0325363 A1
(43) Pub. Date: Oct. 23, 2025

(54) Replacement Heart Valve

(71) Applicant: Medical Equipment Design Innovation, Inc., Irvine, CA (US)

(72) Inventor: Pham Cong Lo, Rancho Mission Viejo, CA (US)

(73) Assignee: Medical Equipment Design Innovation, Inc., Irvine, CA (US)

(21) Appl. No.: 18/641,359

(22) Filed: Apr. 20, 2024

(51) Int. Cl. A61F 2/24; A61L 27/04; A61L 27/06

(52) U.S. Cl. CPC A61F 2/2418; A61F 2/2433; A61F 2/2436; A61L 27/045; A61L 27/06; A61F 2220/0025; A61F 2230/0054

(57) ABSTRACT

A heart valve assembly has a valve frame having a cylindrical body with commissure posts connected to an outflow end, and a leaflet assembly sutured to the plurality of commissure posts and the valve frame. The assembly also has an outflow frame having a plurality of connection posts, with each connection post connected to one of the commissure posts. The valve frame and the annulus frame are made from different materials. A delivery system has a balloon catheter having a shaft, a balloon provided on the shaft adjacent the distal end of the catheter, and a frame seat located on the shaft directly proximal to the balloon. The delivery system includes a sheath assembly having a capsule that slidably covers the balloon and the frame seat such that the outflow frame is seated around the frame seat and the valve frame is seated over the balloon.

### BACKGROUND OF THE INVENTION

### 1. Field of the Invention

The present invention relates to prosthetic heart valves, and in particular, to a transcatheter aortic heart valve prosthesis that is adapted for treating aortic insufficiency (AI), aortic regurgitation (AR), and aortic stenosis (AS) only.

### 2. Description of the Prior Art

Transcatheter heart valves are now commonly used to treat valve disease. These heart valves are typically made of a frame that supports a prosthetic heart valve having leaflets that are usually made of bovine pericardium or porcine pericardium. There are a few types of transcatheter aortic heart valves that are available in the market.

One type is manufactured by Medtronic, which has a frame that is made of a shape memory material such as Nitinol. The frame on this heart valve is self-expanding, so this can be used only with patients having AS where the calcification of the native leaflets in the patient is helpful to anchoring this heart valve. However, this heart valve cannot be used for patients with severe calcification. In addition, this heart valve is not optimal for use with patients without calcification in the AI (Aortic insufficiency) or AR (Aortic regurgitation) conditions because the radial force of the frame is low, which means that the frame can easily migrate from its implanted position. Such a heart valve is also difficult to control after it has been expanded and released from the delivery system.

Another type is manufactured by Edwards Lifesciences and uses a frame that is made of cobalt chromium. Since cobalt chromium is a stiffer material and is not a shape memory material, it needs be expanded by a balloon. Balloon expansion also means that it can only be used with patients that have AS where the calcification of the native leaflets of the patient can help to anchor the implanted heart valve. Unfortunately, this heart valve is also not optimal for use patients that do not exhibit calcification at the aortic valve annulus (i.e., in the AI and AR situations) even though the radial force of the frame is strong. This is because the implanted heart valve can still easily migrate. In addition, when the heart valve is crimped on the balloon and during balloon inflation, the folding of the balloon does not provide a precise implantation at the location of the valve annulus because the balloon unfolds in a rolling mechanism.

### SUMMARY OF THE DISCLOSURE

It is an object of the present invention to provide a transcatheter aortic heart valve prosthesis that can be used for treating AI, AR and AS.

It is another object of the present invention to provide a delivery system for delivering the heart valve prosthesis to the aortic annulus in a patient.

To meet the objectives of the present invention, there is provided a heart valve assembly that has a valve frame having a cylindrical body with an inflow end and an outflow end, a plurality of commissure posts connected to the outflow end, and a leaflet assembly sutured to the plurality of commissure posts and the valve frame. The leaflet assembly has a plurality of leaflets. The assembly also has an outflow frame having a plurality of connection posts, with each connection post connected to one of the plurality of commissure posts. The valve frame and the annulus frame are made from different materials.

The present invention also provides a delivery system having a balloon catheter having a shaft having a distal end, a balloon provided on the shaft adjacent the distal end, and a frame seat located on the shaft directly proximal to the balloon. The delivery system also has a sheath assembly having a capsule that slidably covers the balloon and the frame seat in a manner such that the outflow frame is seated around the frame seat and the valve frame is seated over the balloon.

### BRIEF DESCRIPTION OF THE DRAWINGS

FIG. 1 is a perspective view of a transcatheter aortic heart valve prosthesis according to one embodiment of the present invention shown with the valve leaflets closed.

FIG. 2 is a side view of the heart valve prosthesis of FIG. 1.

FIG. 3 is a perspective view showing only the valve frame and the outflow frame of the heart valve prosthesis of FIG. 1.

FIG. 4 is a side view of the valve frame and the outflow frame of FIG. 3.

FIG. 5 is a side view showing only the valve frame of FIG. 3.

FIG. 6 is a perspective view showing only the valve frame of FIG. 3.

FIG. 7 is a perspective view showing only the outflow frame of FIG. 3.

FIG. 8 is a side view showing only the outflow frame of FIG. 3.

FIG. 9 is a perspective view showing only the valve frame and the leaflet assembly of the heart valve prosthesis of FIG. 1.

FIG. 10 is a perspective view showing only the leaflet assembly of FIG. 9.

FIG. 11 is a perspective view showing only the annulus skirt of FIG. 9.

FIG. 12 is a perspective view showing the combined leaflet assembly and the annulus skirt of FIGS. 10 and 11.

FIG. 13 is a side view if the combined leaflet assembly and the annulus skirt of FIG. 12.

FIG. 14 is a schematic view of a delivery system according to the present invention showing the heart valve prosthesis of FIG. 1 compressed on the balloon, and with the balloon section completely covered by the capsule.

FIG. 15 is a schematic view of the delivery system of FIG. 14 shown with the heart valve prosthesis of FIG. 1 loaded on the balloon, and with the capsule partially withdrawn to partially expose the heart valve prosthesis.

FIGS. 16-18 illustrate the deployment steps of the heart valve prosthesis with the expansion of the balloon and then the withdrawal of the balloon.

FIGS. 19-24 illustrate how the delivery system of FIG. 14 delivers and deploys the heart valve prosthesis of FIG. 1 at the aortic annulus of a human heart.

FIG. 25 is a perspective view of the transcatheter aortic heart valve prosthesis of FIG. 1 shown with a modification made to the outflow frame.

FIG. 26 is a side view of the heart valve prosthesis of FIG. 25.

FIG. 27 is a perspective view showing only the outflow frame of the heart valve prosthesis of FIG. 25.

FIG. 28 is a side view showing only the outflow frame of FIG. 27.

FIG. 29 shows the heart valve prosthesis of FIG. 25 deployed at the aortic annulus of a human heart.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

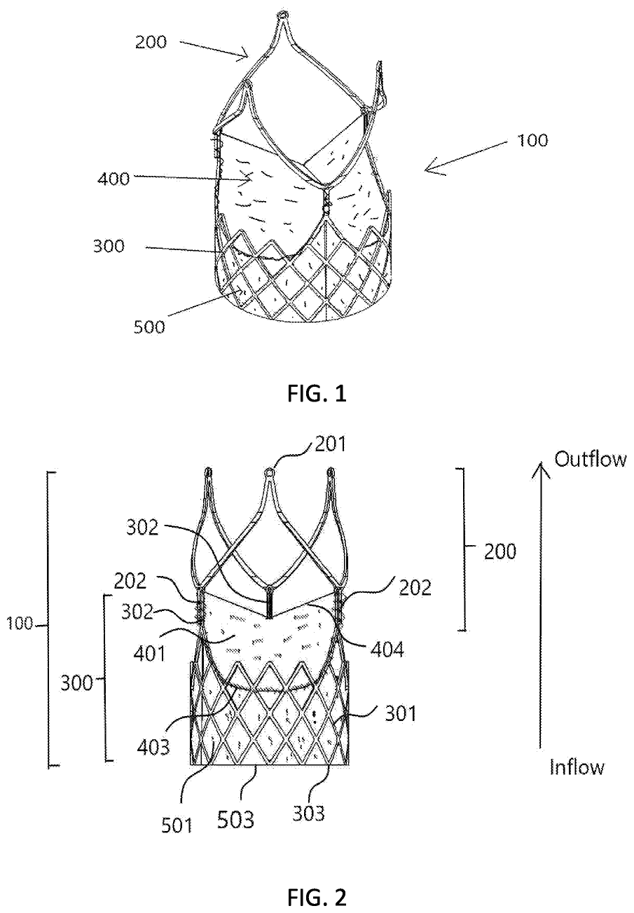

The present invention provides a transcatheter aortic heart valve prosthesis 100 that can be used for treating AI, AR and AS. Referring to FIGS. 1-12, the prosthesis 100 includes a valve frame 300, an outflow frame 200, a tissue leaflet assembly 400 that is retained inside the valve frame 300, and a tissue annulus skirt 500 that is also secured to the valve frame 300. The present invention essentially divides the overall frame of the prosthesis 100 into two separate frames that have two different types of materials that are adapted to provide more effective performance for their intended functions. Specifically, the valve frame 300 is provided in a more rigid material, such as cobalt chromium, so that it can better perform its intended function of securing the prosthesis 100 to the native annulus, while the outflow frame 200 is provided in a more flexible material, such as Nitinol™, so that the outflow frame 200 can assist the valve frame 300 in controlling the balloon 708 so that the balloon 708 does not flare the valve frame 300 during deployment, as will be explained below.

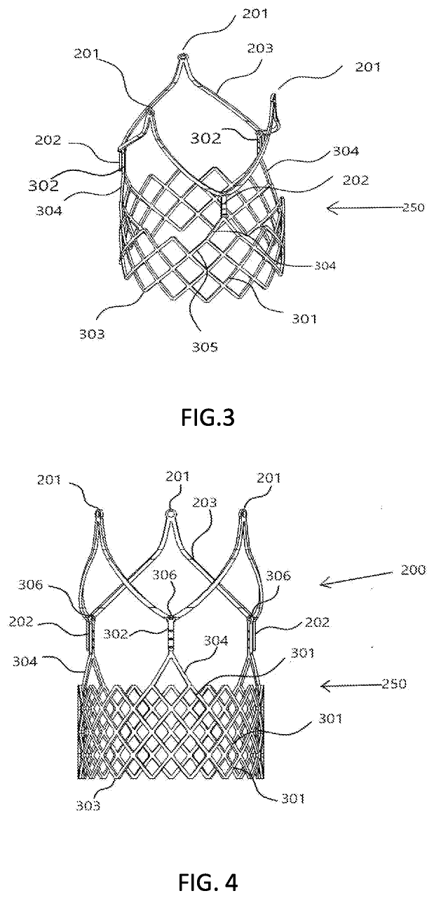

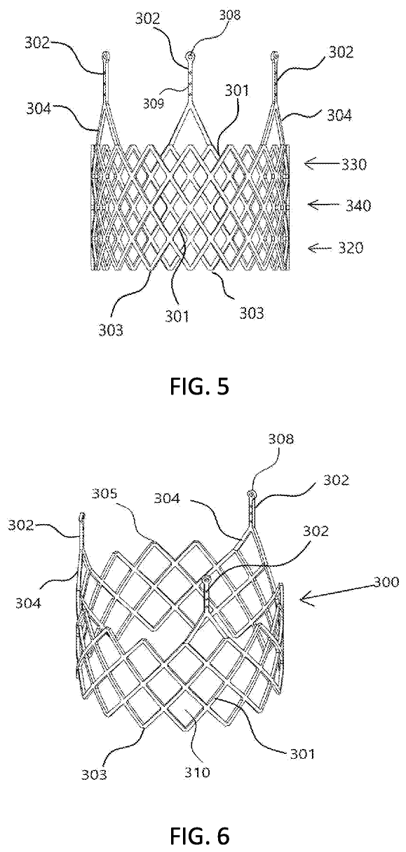

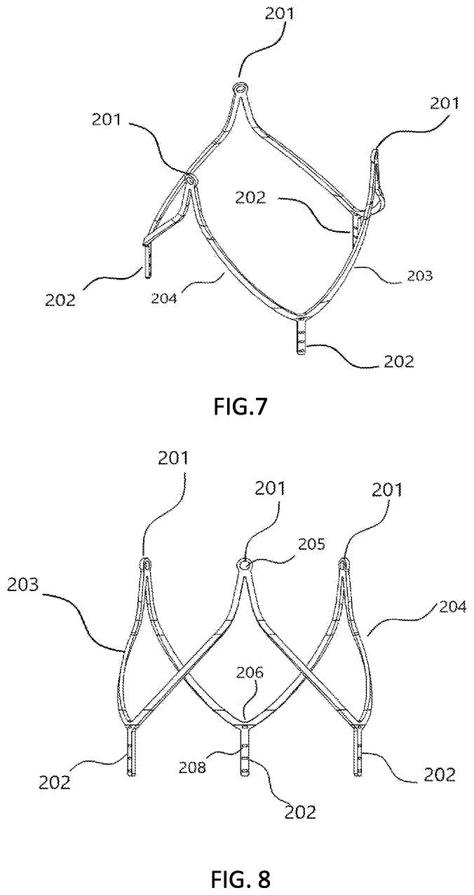

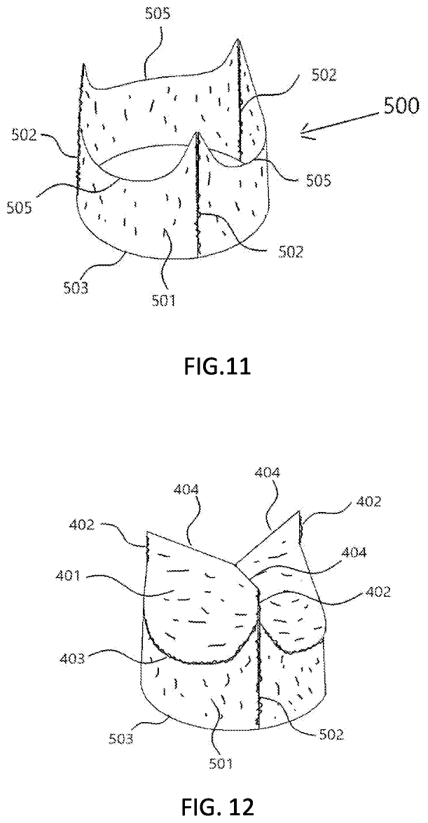

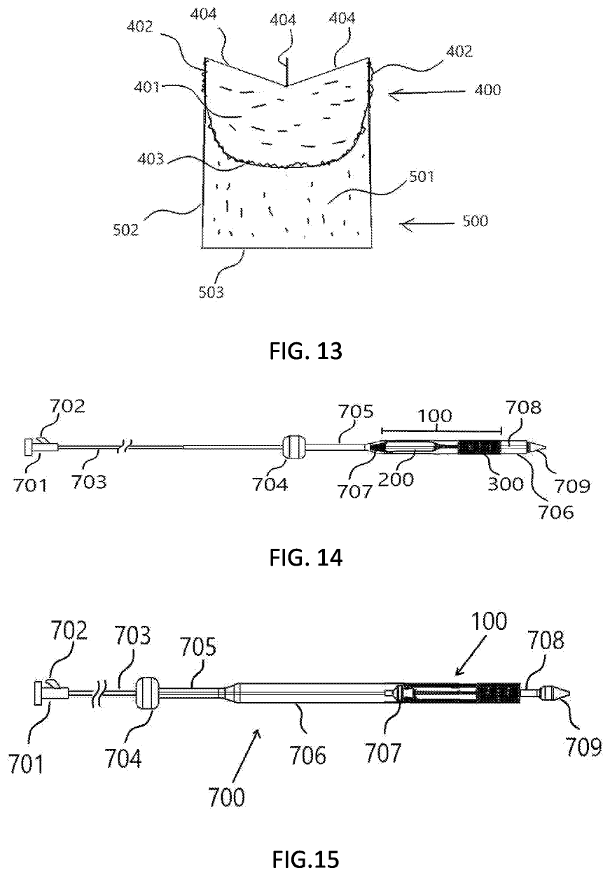

FIGS. 1-2 show the entire prosthesis 100. FIGS. 3, 4 and 9 show the valve frame 300 and the outflow frame 200 and how they are interconnected, with the resulting combined valve frame 300 and outflow frame 200 being designated by the numeral 250. FIGS. 5 and 6 show the valve frame 300 alone, while FIGS. 7 and 8 show the outflow frame 200 alone. FIG. 10 shows the leaflet assembly 400, FIG. 11 shows the annulus skirt 500, and FIGS. 12 and 13 show the combined leaflet assembly 400 and annulus skirt 500.

In a human heart, blood flows from the left ventricle through the aortic valve and towards the aorta. As used herein, the term “inflow side” shall mean the side of the prosthesis 100 from which blood from the left ventricle enters, and the term “outflow side” shall mean the side of the prosthesis 100 where blood exits and flows towards the aorta. These flow directions are shown by arrows “Inflow” and “Outflow” in FIG. 2.

Starting with FIGS. 3-6, the valve frame 300 is preferably made from a more rigid material, such as cobalt chromium, that is mechanically expanded. The valve frame 300 has a generally cylindrical body having three rows of cells 310, with an inflow row 320 and an outflow row 330 separated by a central row 340. Each cell 310 is made up of four struts 301 that define a diamond shape. The inflow row 320 has a plurality of spaced-apart inflow tips 303 defined by the vertices of two adjacent struts 301. The outflow row 330 of cells 310 has a plurality of spaced-apart outflow tips 305 defined by the vertices of two adjacent struts 301. The struts 301 that define the cells 310 for the central row 340 are also used to define the cells 310 for the inflow row 320 and the outflow row 330.

A pair of support struts 304 connect two outflow tips 305 to form a V-shaped support for connecting to one (inflow) end of a commissure post 302. The outflow end of each commissure post 302 has an eyelet 308. In this embodiment, there are three commissure posts 302.

Referring now to FIGS. 3-4 and 7-8, the outflow frame 200 is preferably made from a less rigid and more flexible material, such as a self-expandable material such as Nitinol™, which is preferably self-expandable. The outflow frame 200 has a ring of three V-shaped members 204, with each V-shaped member 204 having two struts 203 that are connected at a vertex 206 that has a connection post 202 extending therefrom. The outflow ends of the two struts 203 of each V-shaped member 204 are connected by a rounded eyelet tip 201. The eyelets 205 formed in each eyelet tip 201 are used for securing the prosthesis 100 to a delivery system, as explained in greater detail below. The V-shaped members 204 define a convex configuration where the ring defined by the vertices 206 has a greater diameter than the diameter of the ring formed by the eyelet tips 201, and where each strut 203 has a curved shape.

As best shown in FIGS. 3-8, the outflow frame 200 is connected to the valve frame 300 by connecting the connection posts 202 to the commissure posts 302. Each connection post 202 has a bump or notch 208 that is adapted to be snap-fitted into a corresponding hole 309 in each commissure post 302. Conversely, notches/bumps can be provided on the commissure posts 302 for fitting into corresponding holes in the connection posts 202. One or more bumps/notches 208 can be provided, and one or more holes 309 can also be provided.

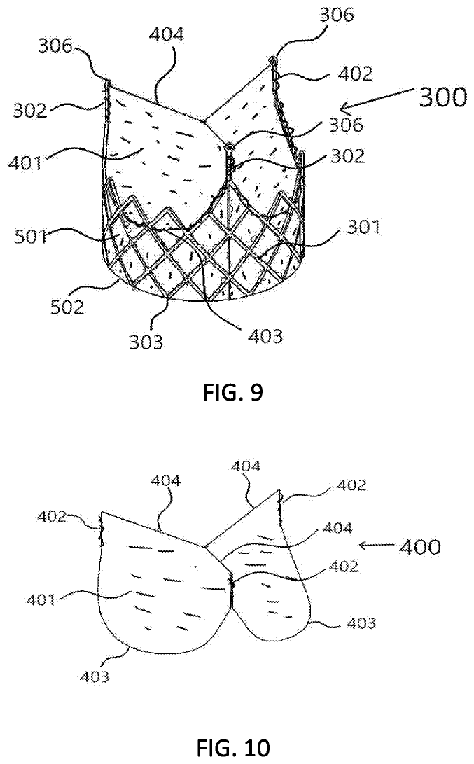

The leaflet assembly 400 is best shown in FIGS. 9, 10, 12 and 13, and has three leaflets 401 that define a center valve coaptation 404. Each leaflet 401 has a commissure edge 402 that is sewn to a corresponding commissure post 302, as best shown in FIG. 9. Each leaflet 401 has another edge 403 that is stitched to a strut 304 and along the zig-zag line for struts 301 of the outflow row of cells 330 (see FIG. 9). The edges 403 define an annular connection edge that is stitched or otherwise connected to the zig-zag line.

The annulus skirt 500 is best shown in FIGS. 9, 11, 12 and 13, and has a skirt body 501 that can be a plurality of (e.g., three) cut pieces of flat tissue material that are stitched along stitch lines 502 to form an annular skirt. The skirt 500 has an annular inflow edge 503 that is aligned with the annular ring formed by the inflow tips 303, and has three curved outflow edges 505, each of which is adapted to be stitched to one edge 403 of the leaflet assembly 400. The edges 403 and 505 have generally the same curvature to facilitate the connection.

The skirt 500 and the leaflets 401 can be made from bovine pericardium or any other conventional tissue that has been treated prior to assembly using known tissue processing techniques (e.g., fixation, etc.). Three leaflets 401 are shown in use although it is also possible to have two leaflets.

The valve frame 300 and the combined leaflet assembly 400 and annulus skirt 500 (see FIG. 9) are usually assembled separately from the outflow frame 200 (see FIGS. 7-8). The combined valve frame 300 and leaflet assembly 400/annulus skirt 500 is then connected to the outflow frame 200 by connecting the connection posts 202 to the commissure posts 302 to arrive at the prosthesis 100 shown in FIGS. 1-2.

The valve frame 300 is preferably made by nickel titanium small tubing (e.g., 7 mm). The design can be created by laser-cutting the tubing and can be shape-set with the desired profile. At under 5 degrees Celsius, the frame becomes elastic and is easy to load onto the balloon 708 at a small size. When the temperature reaches 37 Celsius, the frame returns to its shape-set profile.

The outflow frame 200 can be made by cobalt-chrome small tubing (e.g., 7 mm). The design can be laser-cut on the tubing and expanded like a shaped cylinder with a 23 mm, 26 mm, 29 mm, or 32 mm diameter.

FIGS. 14-18 illustrate a delivery system 700 that is adapted for use in delivering the prosthesis 100 to an aortic annulus, and deploying it at the aortic annulus. The delivery system 700 includes a balloon catheter and a sheath assembly that is sized and configured to ensheath and release the prosthesis 80.

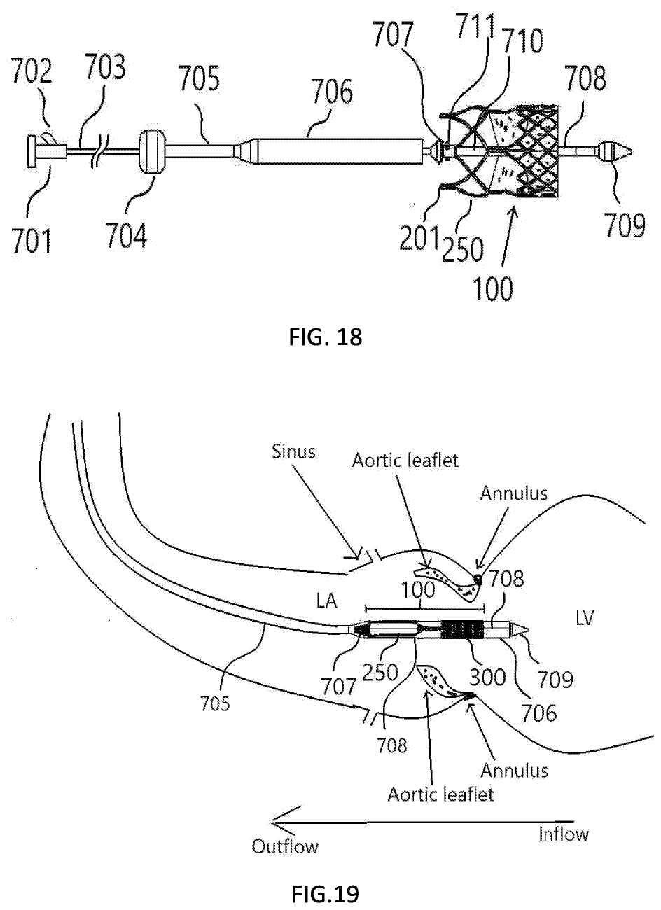

The balloon catheter has a shaft 703 that extends from a T-junction 701 to a tapered distal tip 709. An inflatable balloon 708 is provided on the shaft 703 adjacent the tapered distal tip 709. A frame seat 710 (see FIG. 18) is provided immediately proximal to the balloon 708, which is essentially a portion of the shaft 703. An ear hub 707 is provided on the shaft 703 immediately proximal to the frame seat 710. The ear hub 707 has one or more notches 711 (see FIG. 18) which is adapted to be received by the opening in a corresponding eyelet 205. The remainder of the catheter can be embodied using principles and catheters that are well-known in the art, and will not be described as this is well-known to a person skilled in the art.

The sheath assembly has a hollow outer shaft 705 with a lumen that is sized to receive the shaft 703 of the balloon catheter. A blocking handle 704 is provided at the proximal end of the outer shaft 705 and functions to move the capsule 706 distally (to ensheath the prosthesis 100), and to withdraw the capsule 706 proximally (to release the prosthesis 100). The T-junction 701 can act as a block to limit the proximal travel of the blocking handle 704. A capsule 706 is provided on the distal end of the outer shaft 705. As shown in FIGS. 14 and 17, the blocking handle 704 can extend from adjacent the T-junction 701 to a position along the shaft 703 that is closer to the balloon 708. The shaft 703 is received inside the lumen of the outer shaft 705, and the capsule 706 is adapted to cover the position of the balloon 708 and to be withdrawn proximally to completely expose the balloon 708 and the frame seat 710. As shown in FIG. 14, the prosthesis 100 is positioned at the location of the balloon 708 and the frame seat 710. Specifically, the cobalt chromium valve frame 300 is crimped on to the balloon 708, and the outflow frame 200 is crimped on to the location of the frame seat 710. The sheath assembly is then advanced distally so that the capsule 706 completely covers both the valve frame 300 and the outflow frame 200.

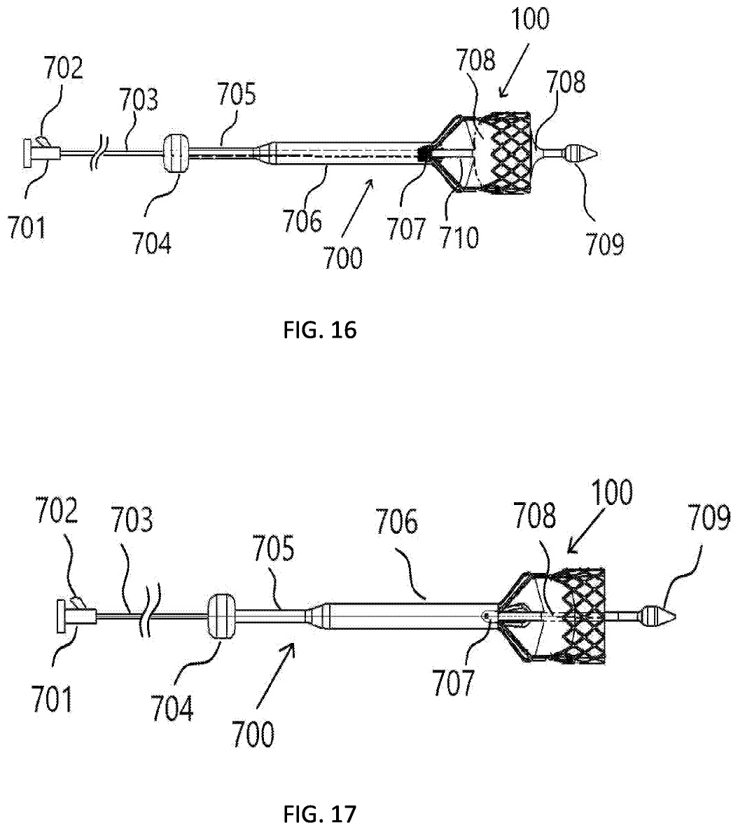

When the prosthesis 100 has been delivered to the location of a native aortic annulus inside a patient, the sheath assembly can be withdrawn so that the capsule 706 is withdrawn such that the distal end of the capsule 706 is at about the location of the ear hub 707 (see FIG. 15), and in this position, the outflow frame 200 begins to self-expand while the valve frame 300 remains crimped. Next, the balloon 708 is inflated (FIG. 16) to expand the valve frame 300. At this point, it can be seen in FIG. 16 that part of the outflow portion of the outflow frame 200 is still retained inside the capsule 706. This detail is important because the inflow portion of the outflow frame 200 that is connected to the valve frame 300 will prevent the valve frame 300 from flaring due to the expansion of the balloon 708. By “flaring”, it is meant that the valve frame 300 can be expanded or flared to assume a curved configuration instead of the generally cylindrical shape shown in FIG. 16. If the valve frame 300 is flared, the leaflet assembly 400 could be damaged.

The sheath assembly is then further withdrawn (FIG. 17) so that the capsule 706 releases the ear hub 707 and the eyelets 205 that are secured to the notch(es) 711 of the ear hub 707. Self-expansion of the eyelets 205 will cause the eyelets 205 to disengage from the notch(es) 711 of the ear hub 707. At this point, balloon expansion has been completed so there is no longer any concern about the further expansion of the balloon 708 causing flaring of the valve frame 300. The balloon 708 is then deflated (FIG. 18) so that the balloon catheter can also be withdrawn.

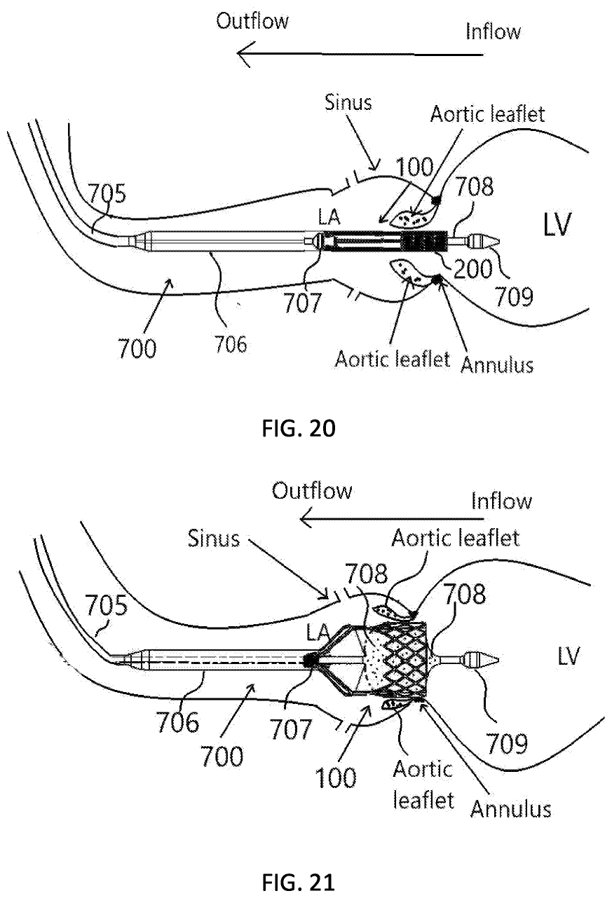

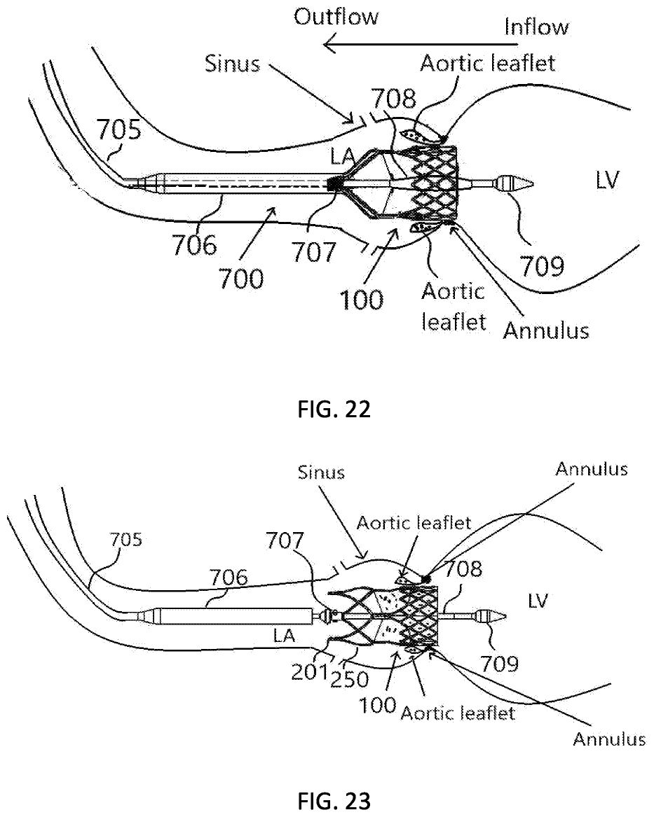

FIGS. 19-24 illustrate how the delivery system 700 delivers and deploys the prosthesis 100 at the aortic annulus of a human heart. Starting with FIG. 19, the delivery system 700 with the prosthesis 100 ensheathed by the capsule 706 is delivered from the aorta through the aortic annulus using transcatheter techniques that are well-known in the art. As shown in FIG. 19, the balloon catheter is preferably positioned so that the valve frame 300 is at the location of the aortic annulus. Next, the capsule 706 is withdrawn such that the distal end of the capsule 706 is at about the location of the ear hub 707 (see FIGS. 15 and 20). In this position, the outflow frame 200 begins to self-expand while the valve frame 300 remains crimped, and then the balloon 708 is inflated (see FIGS. 16 and 21) to expand the valve frame 300. The sheath assembly is then further withdrawn (see FIGS. 17 and 22-23) so that the capsule 706 releases the ear hub 707 and the eyelets 205 that are secured to the notches 711. The balloon 708 is then deflated so that the balloon catheter can also be withdrawn.

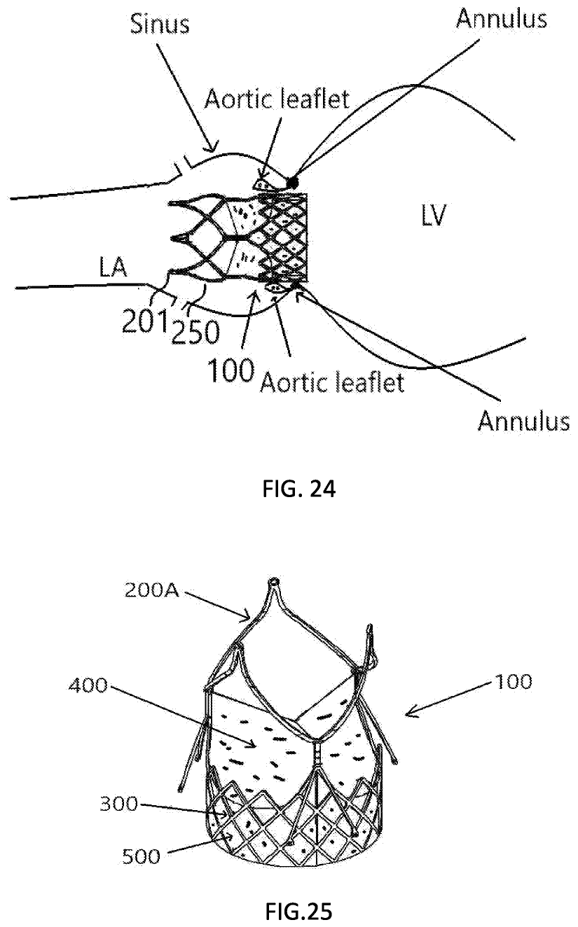

FIG. 24 shows the prosthesis 100 implanted at the aortic annulus. The valve frame 300 is secured at the aortic annulus. For patients with calcification at the location of the aortic annulus, the added mass provided by the calcification will allow the valve frame 300 to be securely fixed to the location of the aortic annulus.

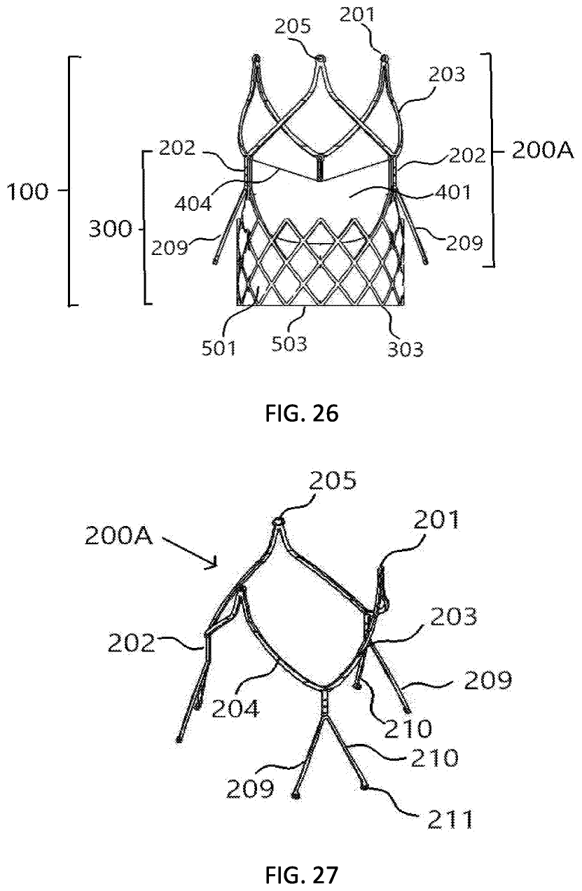

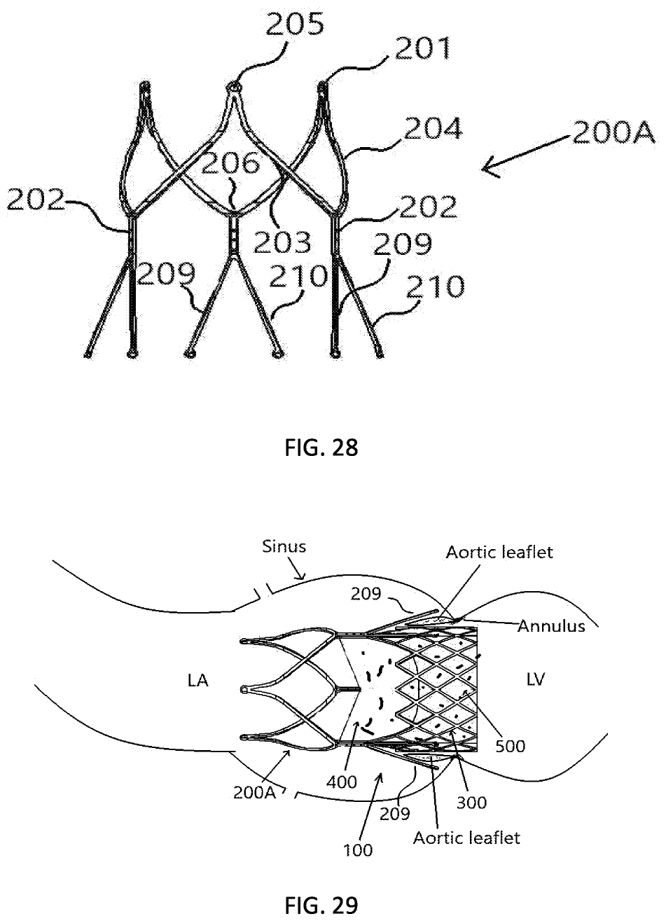

For patients without calcification at the aortic annulus, additional anchoring mechanisms may be needed to securely fix the valve frame 300 at the location of the aortic annulus. FIGS. 25-28 illustrate a modification that can be made to the outflow frame 200, where the modified outflow frame 200A has additional anchor element provided at the inflow side of the outflow frame 200A. Specifically, each anchor element has two legs 209 and 210 that extend in the inflow direction from the inflow end of each connection post 202 to define a V-shaped anchor element. A rounded or ball tip 211 can be provided at then inflow end of each leg 209 and 210 to provide an atraumatic tip to each leg 209 and 210.

FIG. 29 illustrates the modified heart valve prosthesis deployed at the aortic annulus of a human heart. The legs 209 and 210 combine with the body of the valve frame 300 to clip the native aortic leaflets so as to prevent the valve frame 300 from moving in either the inflow or outflow direction.

The present invention provides a number of unique features and benefits.

First, the valve frame 300 is expanded by a balloon with a strong frame radial force, so it can be used for patients with AS (aortic stenosis) at the location of calcification in the aortic annulus and the LVOT.

Second, when the balloon expands the valve frame 300, the embodiment with the anchor elements (legs 209 and 210) can effectively clip the native leaflets to prevent that the prosthesis 100 from moving towards the outflow side, so that the prosthesis can be used for patients with AI (aortic insufficiency) and AR (aortic regurgitation).

Third, since the outflow frame 200 is made of Nitinol™, the frame is easy to shape with the preferred profile so that the balloon 708 does not flare to deform the commissure posts 302. The Nitinol™ outflow frame 200 helps the valve frame 300 to control the balloon 708 so that the balloon 708 does not flare the Cobalt chrome valve frame 300. Thus, the prosthesis is also suitable for use with patients suffering from AS only.

Fourth, the prosthesis 100 allows the procedure to be very precise. Based on the two different materials for the Nitinol™ self-expanding outflow frame 200 and the mechanically-expanded cobalt chromium valve frame 300, when the prosthesis 100 is partially exposed by the capsule 706, the outflow frame 200 slowly self-expands while the valve frame 300 is still crimped on the balloon 708, thereby avoiding a “jump” or sudden expansion by the valve frame 300. This allows the physician time to position the prosthesis 100 and to inflate the balloon 708.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A heart valve assembly, comprising:
a valve frame having a cylindrical body with an inflow end and an outflow end, a plurality of commissure posts connected to the outflow end, and a leaflet assembly sutured to the plurality of commissure posts and the valve frame, the leaflet assembly having a plurality of leaflets;
an outflow frame having a plurality of connection posts, with each connection post connected to one of the plurality of commissure posts; and
wherein the valve frame and the annulus frame are made from different materials.

2. The assembly of claim 1, wherein the outflow frame is made from a self-expanding material and the valve frame is made from a balloon expandable material.

3. The assembly of claim 1, wherein the valve frame is made from cobalt-chromium.

4. The assembly of claim 3, wherein the outflow frame is made from Nitinol.

5. The assembly of claim 1, wherein the outflow frame has a ring of V-shaped of members, with each V-shaped member having two struts that are connected at a vertex, and with one of the connection posts extending from the vertex.

6. The assembly of claim 1, wherein the outflow frame has a ring of V-shaped of members, with each V-shaped member having two struts that are connected at a vertex, and defining an outflow end opposite the vertex, and wherein a rounded eyelet tip is provided at the outflow end of some of the V-shaped members.

7. The assembly of claim 1, wherein the outflow frame has a ring of V-shaped of members that define a convex configuration.

8. A system, comprising:
a heart valve assembly comprising:
a valve frame having a cylindrical body with an inflow end and an outflow end, a plurality of commissure posts connected to the outflow end, and a leaflet assembly sutured to the plurality of commissure posts and the valve frame, the leaflet assembly having a plurality of leaflets;
an outflow frame having a plurality of connection posts, with each connection post connected to one of the plurality of commissure posts; and
wherein the valve frame and the annulus frame are made from different materials;
a delivery system having:
a balloon catheter having a shaft having a distal end, a balloon provided on the shaft adjacent the distal end, and a frame seat located on the shaft directly proximal to the balloon;
a sheath assembly having a capsule that slidably covers the balloon and the frame seat; and
wherein the outflow frame is seated around the frame seat and the valve frame is seated over the balloon.

9. The system of claim 8, wherein:
the outflow frame has a ring of V-shaped of members, with each V-shaped member having two struts that are connected at a vertex, and defining an outflow end opposite the vertex, and wherein a rounded eyelet tip is provided at the outflow end of some of the V-shaped members
an ear hub provided on the shaft immediately proximal to the frame seat; and
the eyelet engages the ear hub when the outflow frame is seated around the frame seat and the valve frame is seated over the balloon.

10. The system of claim 8, wherein the outflow frame is made from a self-expanding material and the valve frame is made from a balloon expandable material.

11. The system of claim 8, wherein the valve frame is made from cobalt-chromium.

12. The system of claim 11, wherein the outflow frame is made from Nitinol.

13. A method of delivering a heart valve assembly to an aortic annulus which has native aortic leaflets, comprising:
providing a heart valve assembly comprising:
a valve frame having a cylindrical body with an inflow end and an outflow end, a plurality of commissure posts connected to the outflow end, and a leaflet assembly sutured to the plurality of commissure posts and the valve frame, the leaflet assembly having a plurality of leaflets;
an outflow frame having a plurality of connection posts, with each connection post connected to one of the plurality of commissure posts; and
wherein the valve frame and the annulus frame are made from different materials;
providing a delivery system having:
a balloon catheter having a shaft having a distal end, a balloon provided on the shaft adjacent the distal end, and a frame seat located on the shaft directly proximal to the balloon;
a sheath assembly having a capsule; and
crimping the heart valve assembly on to the balloon catheter by seating the outflow frame around the frame seat and the valve frame over the balloon;
sliding the capsule to cover the balloon and the frame seat;
advancing the delivery system with the heart valve assembly ensheathed by the capsule to the location of an aortic annulus in a human heart with the valve frame at the location of the aortic annulus;
withdrawing the capsule such that the capsule does not cover the outflow frame, and such that the outflow frame begins to self-expand while the valve frame remains crimped;
inflating the balloon to expand the valve frame;
deflating the balloon; and
withdrawing the balloon catheter.

14. The method of claim 13, further including:
providing the outflow frame with a ring of V-shaped of members, with each V-shaped member having two struts that are connected at a vertex, and defining an outflow end opposite the vertex, and wherein a rounded eyelet tip is provided at the outflow end of some of the V-shaped members;
providing an ear hub on the shaft immediately proximal to the frame seat; and
wherein the eyelet engages the ear hub when the valve frame is seated around the frame seat and the annulus frame is seated over the balloon.

15. The method of claim 13, further including providing the outflow frame as a self-expanding material and the valve frame as a balloon expandable material.

16. The method of claim 13, wherein the valve frame is made from cobalt-chromium.

17. The method of claim 16, wherein the outflow frame is made from Nitinol.
